(19) United States
(12) Patent Application Publication
(10) Pub. No.: US 2025/0152003 A1
(43) Pub. Date: May 15, 2025

(54) MEASURING BIOMECHANICAL AND BIREFRINGENT PROPERTIES OF EYE TISSUE

(71) Applicant: Alcon Inc., Fribourg (CH)

(72) Inventors: Gangjun Liu, Portland, OR (US); Lingfeng Yu, Rancho Santa Margarita, CA (US); Yilin Zhao, Irvine, CA (US)

(21) Appl. No.: 18/943,137

(22) Filed: Nov. 11, 2024

Related U.S. Application Data
(60) Provisional application No. 63/598,684, filed on Nov. 14, 2023.

(51) Int. Cl. A61B 3/10

(52) U.S. Cl. CPC A61B 3/102

(57) ABSTRACT

In certain embodiments, an ophthalmic diagnostics system includes a polarization sensitive optical coherence tomography (PS-OCT) device. The system also includes a memory having executable instructions. The system also includes a processor in communication with the memory and configured to execute the instructions to cause the PS-OCT device to emit polarized light to a measurement location on eye tissue of a patient and generate PS-OCT data based on the polarized light. The processor is further configured to execute the instructions to determine birefringent information for the measurement location based on the PS-OCT data and to determine biomechanics information for the measurement location based on the PS-OCT data. The processor is further configured to execute the instructions to determine a correlation between at least a portion of the biomechanics information and at least a portion of the birefringent information.

### PRIORITY CLAIM

This application claims the benefit of priority of U.S. Provisional Patent Application Ser. No. 63/598,684, titled “MEASURING BIOMECHANICAL AND BIREFRINGENT PROPERTIES OF EYE TISSUE” filed on Nov. 14, 2023, whose inventors are Gangjun Liu, Lingfeng Yu, and Yilin Zhao, all of which are hereby incorporated by reference in their entirety as though fully and completely set forth herein.

### BACKGROUND

Tissue biomechanics, such as corneal biomechanics, can play an important role in understanding, diagnosing, and treating eye diseases such as glaucoma, Keratoconus, and ectasia. The ability to measure such biomechanics and correlate them to other information, however, is often restricted by hardware limitations.

### SUMMARY

The present disclosure relates to diagnostic systems and methods, and more particularly, to systems and methods for measuring biomechanical and birefringent properties of eye tissue.

In certain embodiments, one general aspect includes an ophthalmic diagnostics system. The ophthalmic diagnostics system includes a polarization sensitive optical coherence tomography (PS-OCT) device. The system also includes a memory having executable instructions. The system also includes a processor in communication with the memory and configured to execute the instructions to cause the PS-OCT device to emit polarized light to a measurement location on eye tissue of a patient and generate PS-OCT data based on the polarized light. The processor is further configured to execute the instructions to determine birefringent information for the measurement location based on the PS-OCT data and to determine biomechanics information for the measurement location based on the PS-OCT data. The processor is further configured to execute the instructions to determine a correlation between at least a portion of the biomechanics information and at least a portion of the birefringent information.

In certain embodiments, another general aspect includes a method of operating an ophthalmic diagnostics system including a polarization sensitive optical coherence tomography device (PS-OCT) device. The method includes emitting, by the ophthalmic diagnostics system, polarized light to a measurement location on eye tissue of a patient. The method also includes generating, by the ophthalmic diagnostics system, PS-OCT data for the measurement location based on the polarized light. The method also includes determining, by the ophthalmic diagnostics system, birefringent information for the measurement location based on the PS-OCT data. The method also includes determining, by the ophthalmic diagnostics system, biomechanics information for the measurement location based on the PS-OCT data. The method also includes determining, by the ophthalmic diagnostics system, a correlation between at least a portion of the biomechanics information and at least a portion of the birefringent information.

### BRIEF DESCRIPTION OF THE DRAWINGS

So that the manner in which the above-recited features of the present disclosure can be understood in detail, a more particular description of the disclosure, briefly summarized above, may be had by reference to embodiments, some of which are illustrated in the appended drawings. It is to be noted, however, that the appended drawings illustrate only exemplary embodiments and are therefore not to be considered limiting of its scope, and may admit to other equally effective embodiments.

FIG. 1A illustrates an example configuration of an ophthalmic diagnostics system, according to certain embodiments of the present disclosure.

FIG. 1B illustrates another example configuration of an ophthalmic diagnostics system, according to certain embodiments of the present disclosure.

FIG. 2 is a block diagram of various components of the ophthalmic diagnostics system of FIGS. 1A-B, according to certain embodiments of the present disclosure.

FIG. 3 illustrates an example configuration of a polarization sensitive optical coherence tomography device, according to certain embodiments of the present disclosure.

FIG. 4 illustrates an example of a process for operating an ophthalmic diagnostics system in response to a stimulus, according to certain embodiments of the present disclosure.

FIG. 5 illustrates an example of a process for operating an ophthalmic diagnostics system without an external stimulus from a stimulation device, according to certain embodiments of the present disclosure.

To facilitate understanding, identical reference numerals have been used, where possible, to designate identical elements that are common to the figures. It is contemplated that elements and features of one embodiment may be beneficially incorporated in other embodiments without further recitation.

### DETAILED DESCRIPTION

For the purposes of promoting an understanding of the principles of the disclosure, reference will now be made to the implementations illustrated in the drawings, and specific language will be used to describe the same. It will nevertheless be understood that no limitation of the scope of the disclosure is intended. Any alterations and further modifications to the described systems, devices, instruments, methods, and any further application of the principles of the present disclosure are fully contemplated as would normally occur to one skilled in the art to which the disclosure relates. In particular, the features, components, and/or steps described with respect to one implementation may be combined with the features, components, and/or steps described with respect to other implantations of the disclosure. For simplicity, in some instances the same reference numbers are used throughout the drawings to refer to the same or like parts.

Optical coherence tomography (OCT) is an interferometric analysis technique for structural examination of a sample that is at least partially reflective to light, such as a biological tissue. OCT systems may be used to determine distance and depth profiles and other information based on interference patterns created by the interaction between a reflected beam from a reference reflector and a reflected beam from a sample.

In an OCT system, a single OCT source beam is split, by a beam splitter, into two component beams: a sample beam that is propagated to and at least partially reflected by a sample, and a reference beam that is propagated to and reflected by a reference reflector. Each component beam is typically reflected back to the beam splitter and combined, although certain OCT systems may not require each reflected beam to return to the beam splitter to be combined. When the reflected sample beam and reflected reference beam are combined, an interference pattern is generated, which may be used to measure distances and depth profiles of the sample and other information and to image internal target structures that the sample beam passed through. In ophthalmic surgery, an OCT system may be used, for example, to provide cross sectional views of the retina in high resolution.

OCT systems generally rely on the intensity of backscattered reflected light. These OCT systems cannot directly differentiate between different types of tissues and have trouble distinguishing between layers of tissues. This is especially problematic when an ocular disease has led to damaged, distorted, displaced, or disrupted retinal layers. Thus, any additional tissue discrimination provided by polarization sensitive optical coherence tomography (PS-OCT) is of significant value in the diagnosis of ocular diseases, planning of surgical treatments, and performance of such treatments.

PS-OCT is a functional imaging method that is an extension of OCT. PS-OCT provides additional contrast and tissue discrimination (in relation to traditional OCT methods that only measure reflectivity) by additionally using the light polarizing properties of a sample to discern further information relating to the sample. The sample may be a biological sample, such as a human eye. PS-OCT is based on the concept that some tissues can change the polarization state of incident light. PS-OCT may be used to visualize certain polarization properties of the eye, for instance birefringence, diattenuation, and depolarization. Because several structures or layers of the eye, including the cornea, retinal epithelium and retinal nerve fiber layer, alter the polarization state of incident light, a PS-OCT image (also referred to herein as a pictorial representation) can present tissue-specific contrast, allowing the user to determine polarization properties such as fiber alignment and fiber density.

PS-OCT may be used to measure tissue birefringence. Generally, tissue birefringence is the optical property of a material having a refractive index (light propagation velocity) that depends on the polarization and propagation direction of light. In PS-OCT, tissue birefringence may be determined by measuring the phase retardation of the light incident upon and reflected from the sample, as a function of tissue depth. In a sample, tissue birefringence is often caused by narrow fibrous structures that cannot be resolved by conventional OCT systems. Such OCT systems only account for the intensity of reflected or backscattered light.

PS-OCT, in contrast to conventional OCT, can utilize information carried by polarized light in addition to information carried by reflected light, thereby providing enhanced tissue discrimination and quantitative measurements of sample properties. To measure the polarizing properties of a sample using PS-OCT, the sample is typically illuminated with light of a known polarization state, or illuminated with light at multiple polarization states with known relations. For example, the sample may be illuminated by a bulk optics system or light generated by a PS-OCT source may be propagated to the sample via an optical fiber, which may be a polarization maintaining (PM) fiber. When a PS-OCT light source is used, it generates a PS-OCT source beam, which is first propagated through a polarizing filter of a known polarization. The polarized PS-OCT source beam is then split by a beam splitter into two component beams, a sample beam that is propagated to and at least partially reflected by a sample (the sample arm), and a reference beam that is propagated to and reflected by a reference reflector (the reference arm).

Because PS-OCT is polarization sensitive, each component beam may pass through or contact certain wave plates, filters, mirrors, or lenses, depending on the specific PS-OCT configuration. After each component beam is respectively reflected by the sample and the reference reflector, they may be recombined at the beam splitter to form a reflected PS-OCT beam. When the reflected sample beam and the reflected reference beam are recombined, an interference pattern is formed. The reflected PS-OCT beam is directed to a polarization sensitive detector, which detects and generates data relating to the reflected polarized light of the reflected PS-OCT beam. The data may be transmitted to and used by a processor to determine certain polarization properties, such as fiber orientation, of the tissue reflecting the sample beam.

Although PS-OCT can be used to measure tissue birefringence, the relationship between tissue birefringence and tissue biomechanics is difficult to identify using available hardware in typical PS-OCT systems. Eye-tissue biomechanics, including corneal biomechanics such as corneal stiffness, can play an important role in understanding, diagnosing, and treating diseases such as glaucoma, Keratoconus, and ectasia. Detailed clinical assessment of corneal biomechanics has the potential to revolutionize the ophthalmic industry by providing, for example, personalized LASIK (Laser-Assisted in Situ Keratomileusis) and cataract surgery.

Assessment and understanding of corneal biomechanics has application in corneal surgery for determining patient suitability and improving the safety and efficacy of surgery procedures. For example, corneal ectasia is a very rare but serious complication of refractive procedures that arises in 0.04-0.6% of cases. Measuring the corneal biomechanics can facilitate preoperative screening of refractive surgery candidates so as to minimize the risk of postoperative corneal ectasia. In cataract surgery, for example, surgically induced astigmatism (SIA) ranges from 0 to 1.5 D can be a significant source of refractive surprises. Accurate measurement of corneal biomechanics can enable better prediction of patient-specific SIA for cataract surgery and increase the surgery outcomes.

The present disclosure describes examples of systems and methods for determining biomechanics information and birefringent information for an eye of a patient based on PS-OCT data such as PS-OCT images. In various embodiments, the PS-OCT data can be used to reveal birefringent properties of eye tissue and, additionally, to characterize corneal biomechanical properties using methods such as phase-decorrelation OCT. For example, the PS-OCT data can enable identification of differences in collagen fiber distributions between healthy and keratoconic corneas. Advantageously, in certain embodiments, utilizing the PS-OCT data to generate both birefringent and biomechanics information can eliminate a need for additional hardware. In addition, or alternatively, in various embodiments, the biomechanics information and the birefringent information can be correlated and used for improved diagnosis and treatment of diseases such as glaucoma, Keratoconus, and ectasia. Particular examples will be described in greater detail relative to the Drawings.

For illustrative purposes, the present disclosure periodically describes various examples in relation to corneas. However, it should be appreciated that similar principles are applicable to measuring properties for other portions of the eye and/or other tissue.

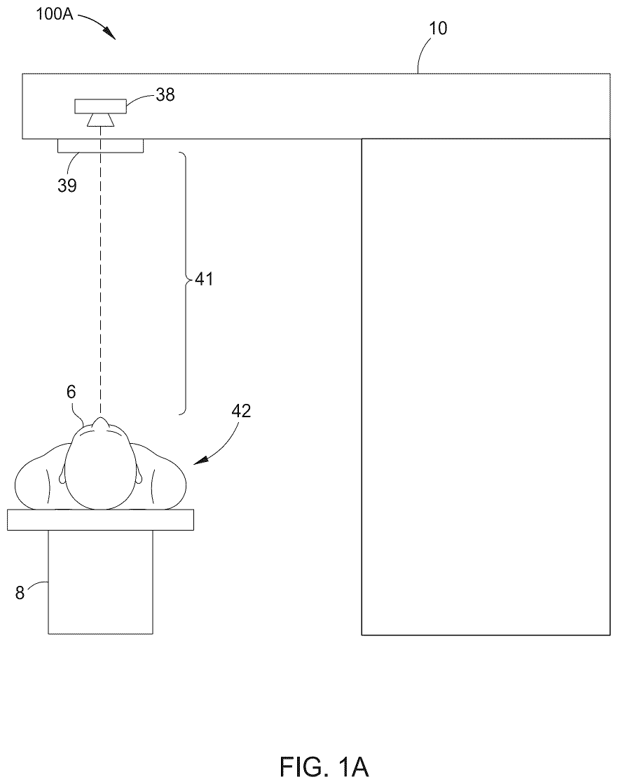

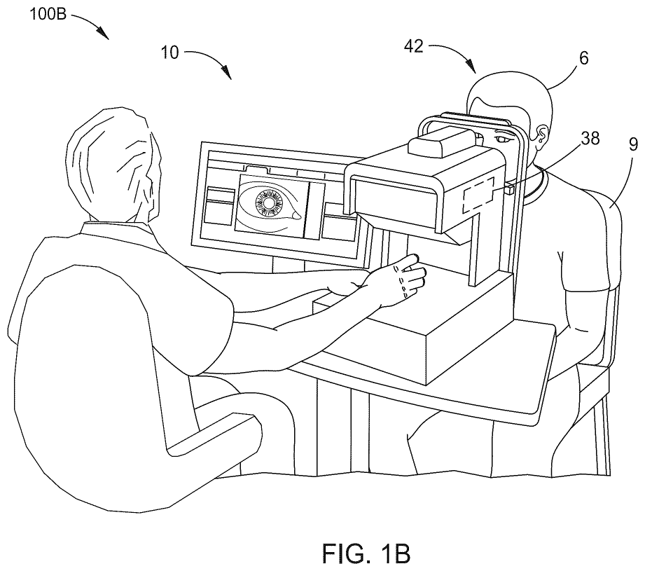

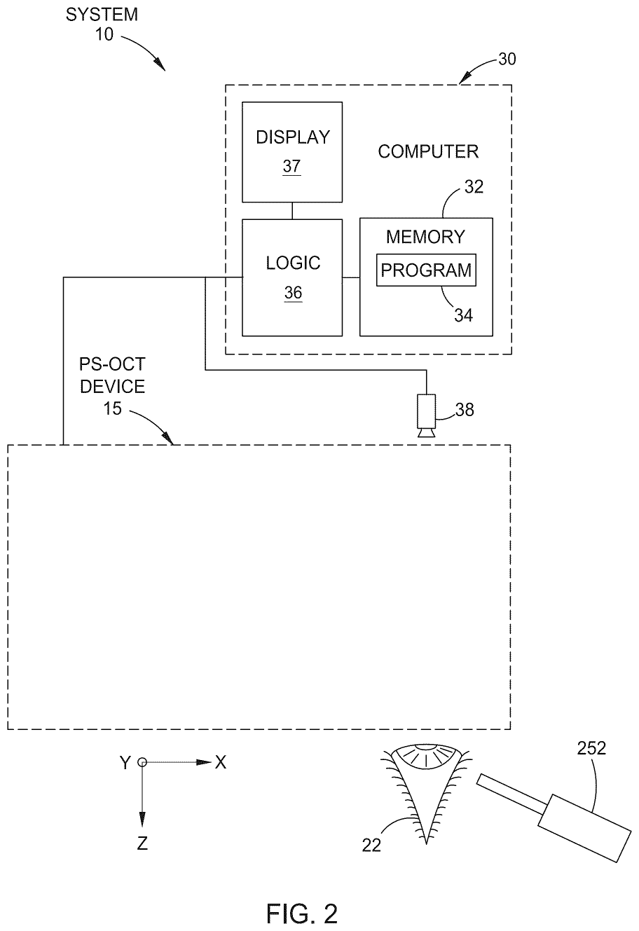

FIGS. 1A, 1B, and 2 illustrate an example of an ophthalmic diagnostics system 10 according to certain embodiments. The ophthalmic diagnostics system 10 may be used for different types of diagnostic and treatment procedures. For example, the ophthalmic diagnostics system 10 may be used for diagnosis or treatment of glaucoma, Keratoconus, and/or ectasia. In addition, or alternatively, the ophthalmic diagnostics system 10 may be used to provide data for personalizing LASIK or cataract surgery.

FIG. 1A shows a configuration 100A of the ophthalmic diagnostics system 10. In particular, FIG. 1A illustrates a head 6 of a patient 42 lying on a bed 8. In the illustrated example, the ophthalmic diagnostics system 10 includes a camera 38 and a part 39 where multiple imaging light beams can exit the ophthalmic diagnostics system 10 and travel through an area 41 towards the patient 42.

FIG. 1B shows a configuration 100B of the ophthalmic diagnostics system 10. In the configuration 100B, the ophthalmic diagnostics system 10 is configured as a desktop imaging system in which the patient 42 sits in a chair 9.

With reference to FIG. 2, the ophthalmic diagnostics system 10 includes a PS-OCT device 15, the camera 38, and a control computer 30, coupled as shown. In some embodiments, the ophthalmic diagnostics system 10 also includes a stimulation device 252. The computer 30 includes logic 36, a memory 32 (which stores a computer program 34), and a display 37, coupled as shown. For ease of explanation, the following xyz-coordinate system is used: The z-direction is defined by the propagation direction of the imaging light beams, and the xy-plane is orthogonal to the propagation direction. Other suitable xyz-coordinate systems may be used.

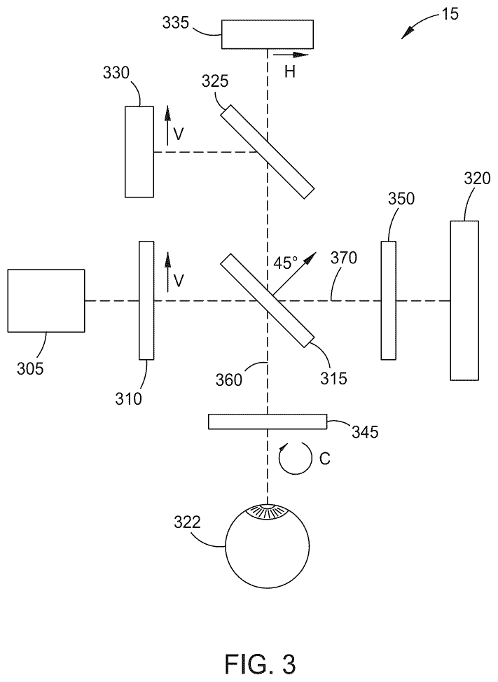

The PS-OCT device 15 can generate and emit polarized light to tissue of an eye 22 of the patient 42. For example, the polarized light may be guided to a suitable location, for example, on a corneal surface of the eye 22. The PS-OCT device 15 can include, for example, multiple polarization detectors such as, for example, a vertical polarization sensitive detector and a horizontal polarization sensitive detector. In certain embodiments, the PS-OCT device 15 can thereby generate and provide PS-OCT data. The PS-OCT data can be multichannel in nature, for example, by including data generated by each of the vertical polarization sensitive detector and the horizontal polarization sensitive detector. An example of the PS-OCT device 15 will be described in greater detail relative to FIG. 3.

The camera 38 can continuously capture one or more images of the patient 42. For example, the camera 38 can be focused on the eye 22. Examples of the camera 38 include a video, interferometry, thermal imaging, ultrasound, OCT, and eye-tracking cameras. The camera 38 delivers image data, which represent recorded images of the eye 22, to the computer 30. In some embodiments, the camera 38 can be an integral part of the PS-OCT device 15, rather than separate as illustrated in FIG. 2.

The stimulation device 252 can be any suitable device for applying an external stimulus to the eye 22. For example, the stimulation device 252 may apply an air pulse to induce a corneal displacement. In another example, the stimulation device 252 may apply an ultrasound wave to induce a corneal displacement. In other examples, the stimulation device 252 can be a laser or a physical actuator. Other examples of stimulation will be apparent to one skilled in the art after a detailed review of the present disclosure. In some embodiments, the stimulation device 252 may be omitted.

The computer 30 controls components of the ophthalmic diagnostics system 10 in accordance with the computer program 34. For example, the computer 30 controls PS-OCT device 15 to emit the polarized light to a desired measurement location on the eye 22, such as a desired measurement location on a corneal surface thereof. In certain embodiments, the computer 30 can receive PS-OCT data from the PS-OCT device 15 and, based thereon, determine corneal biomechanics information and corneal birefringent information for the eye 22 in real-time for the desired measurement location. The memory 32 stores information used by the computer 30. For example, memory 32 may store images of the eye 22, PS-OCT data, and/or other suitable information, and the computer 30 may access information from the memory 32. In various embodiments, the computer program 34 and its functionality, such as the focusing of the imaging light beams, can be directed by a user such as a medical professional.

In certain embodiments, the ophthalmic diagnostics system 10 can operate in response to stimulation, for example, from the stimulation device 252. In various embodiments, the computer 30 of FIG. 2 monitors for an indication of a stimulus applied to the eye 22 (e.g., to a corneal surface thereof), for example, by the stimulation device 252. When the computer 30 detects a stimulus applied by the stimulation device 252, or is notified of such as stimulus (e.g., by a user), the computer 30 can instruct, or cause, the PS-OCT device 15 to emit polarized light to a measurement location on the eye 22 (e.g., the cornea). The computer 30 can then receive, from the PS-OCT device 15, PS-OCT data resultant from the polarized light, as described above and in more detail relative to FIG. 3.

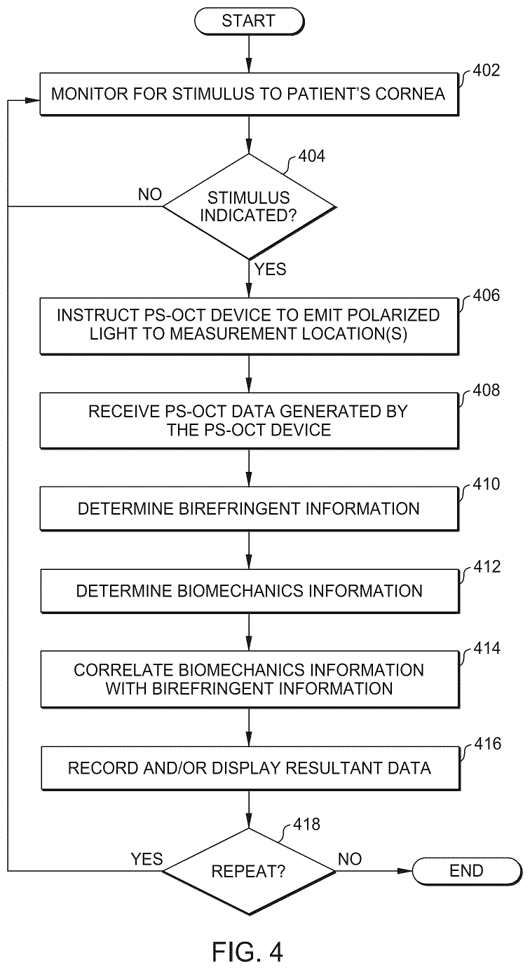

Still with reference to operation of the ophthalmic diagnostics system 10 in response to stimulation, the PS-OCT data received by the computer 30 can be used to determine, for example, corneal biomechanics information and corneal birefringent information for the measurement location. The corneal biomechanics information that is determined can include, for example, vibration amplitude, vibration frequency, a quantification of tissue stiffness (e.g., Young's modulus), combinations of the foregoing and/or the like. Advantageously, in certain embodiments in which the PS-OCT data is multichannel in nature, for example, by including data generated by multiple polarization sensitive detectors as described previously, the computer 30 can separately process each channel of the PS-OCT data and determine separate corneal biomechanics for each channel (e.g., a separate vibration amplitude, a separate vibration frequency, a separate Young's modulus, etc.). The computer 30 can record the PS-OCT data, the corneal biomechanics information, the corneal birefringent information, and/or related data in the memory 32 or other storage. Further examples of the ophthalmic diagnostics system 10 operating in response to stimulation will be described relative to FIG. 4.

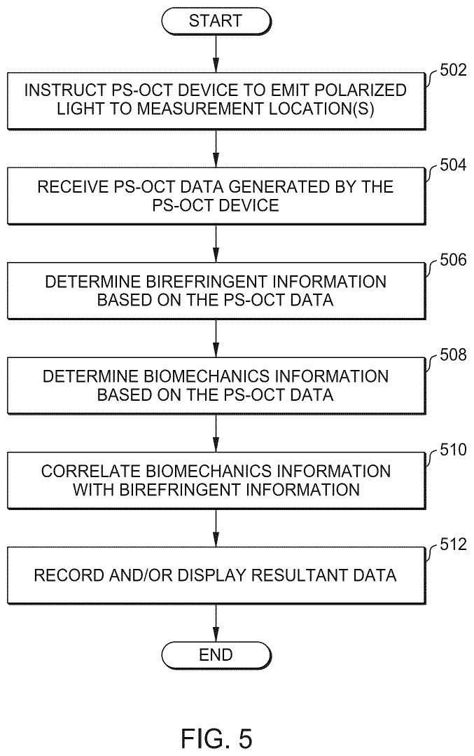

In addition, or alternatively, in certain embodiments, the ophthalmic diagnostics system 10 can operate passively without an external stimulus from a stimulation device such as the stimulation device 252. In various embodiments, the computer 30 can instruct, or cause, the PS-OCT device 15 to emit polarized light to a measurement location on the eye 22 (e.g., the cornea). The computer 30 can then receive, from the PS-OCT device 15, PS-OCT data resultant from the polarized light, as described in more detail relative to FIG. 3. The PS-OCT data can be used by the computer 30 to determine, for example, corneal biomechanics information and corneal birefringent information for the measurement location. The corneal biomechanics can be determined, for example, based on phase decorrelation or intensity variance. The computer 30 can record the PS-OCT data, the corneal biomechanics information, the corneal birefringent information, and/or related data in the memory 32 or other storage. Further examples of the ophthalmic diagnostics system 10 operating without an external stimulus from a stimulation device will be described relative to FIG. 5.

FIG. 3 is a schematic diagram of PS-OCT device 15. As shown, PS-OCT device 15 includes PS-OCT light source 305, polarization component 310, beam splitter 315, reference reflector 320, polarizing beam splitter (PBS) 325, vertical polarization sensitive detector 330, horizontal polarization sensitive detector 335, and wave plate 345. In FIG. 3, PS-OCT device 15 also includes a polarization system 350.

The PS-OCT light source 305 generates a PS-OCT source beam, which is propagated through polarization component 310, to a beam splitter 315. Polarization component 310 may control a polarization of the PS-OCT source beam. As shown, polarization component 310 is a vertical polarizer, and polarization system 350 is a quarter wave plate (“QWP”) positioned at 22.5 degrees (described in detail below). The PS-OCT light source may be, for example, a superluminescent diode, a supercontinuum laser, or a swept-source laser. Although labelled as a PS-OCT light source, PS-OCT light source 305 may be any light source suitable for OCT imaging. In device 15, any beam of light described may be propagated by optical fibers. For example, light may be propagated by a conventional (non-PM) single-mode fiber and a modulator may be implemented to differentiate between the polarization changes in the tissue and the system. Alternatively, the light may be propagated by a polarization maintaining (PM) fiber, which allows the light to be propagated in two linear orthogonal channels (fast and slow). Each linear channel of orthogonal light is generally perpendicular to the other (i.e., the fast channel is perpendicular to the slow channel).

Upon leaving PS-OCT light source 305, the PS-OCT source beam is typically polarized light, which means that it is light generally oscillating in one single plane. In contrast, unpolarized light is light that is oscillating in more than one plane. When the PS-OCT source beam passes through polarization component 310, the transmitted light that reaches beam splitter 315 is vertically polarized, meaning the light is only oscillating in a vertical direction. Polarization component 310 is a type of linear polarizer, another example of which is a horizontal polarizer, in which the transmitted light is only oscillating in a horizontal direction. As applied to linearly polarized light, the terms “vertical” and “horizontal” respectively refer to the direction the wave oscillates in the optical axis in which it is propagated. A linear polarizer may be an absorptive polarizer, which absorbs the unwanted polarization states and transmits the chosen polarization state. For example, a vertical polarizer absorbs unwanted polarization states, including horizontal polarization states, and only transmits vertical polarization states. A linear polarizer may alternatively be a beam-splitting polarizer, which splits the unpolarized light into two component beams, each with an opposite polarization states (i.e., one component beam would have a vertical polarization state and the other, a horizontal polarization state).

Although polarization component 310 is described in FIG. 3 as being a vertical polarizer, polarization component 310 may alternatively be a horizontal polarizer or other polarizer of a known polarization state, although using a vertical or horizontal polarizer may facilitate analysis of data generated relating to a reflected PS-OCT beam. In certain embodiments, the role of polarization component 310 is to receive the incident PS-OCT source beam and transmit a polarized PS-OCT source beam with a known polarization. Similarly, the importance of wave plate 345 and polarization system 350 is to receive an incident beam of light and transmit a beam of light with a known polarization, described in detail below. Accordingly, some PS-OCT systems may not include polarization system 350 depending on the system's configuration. For example, polarization system 350 may be not be used when the beam of light leaving the beam splitter on the reference arm is orthogonal, (e.g., the light leaving beam splitter 315 is at a 45 degree angle and wave plate 345 is oriented at 0 degrees with respect to the vertical or horizontal polarization of the light propagated on the sample beam).

When the vertically polarized PS-OCT source beam reaches the beam splitter, it is split into two component beams, a polarized sample beam that is propagated to and at least partially reflected by a sample, and a polarized reference beam that is propagated to and reflected by a reference reflector. The sample beam may be referred to as propagated on sample arm 360, and the reference beam may be referred to as propagated on reference arm 370. As described in detail below, the polarization state of the reference beam is known because optical properties of the reference reflector are known and because the polarization state of the reference beam is known. In FIG. 3, the polarization state of the reference beam is known because the reference beam is converted by means of a polarization system 350, prior to reaching reference reflector, to produce two orthogonal polarization components with equal intensities upon re-entry of the beam splitter. Polarization system 350 may be a QWP, for example, a QWP at 22.5 degrees. Each component beam is typically reflected back to the beam splitter 315 and combined, although certain PS-OCT systems may not require each reflected beam to return to the beam splitter to be combined.

When the sample beam is transmitted along sample arm 360, it passes through wave plate 345 before reaching sample 322. As shown, wave plate 345 is a QWP and sample 322 is a human eye. Wave plate 345 converts the sample beam's linear vertically polarized light to circularly polarized light with a known polarization state. Although PS-OCT device 15 is described as using QWP's, other systems may implement other types of wave plates, for instance a half-wave plate (“HWP”) or a full wave plate (“FWP”). A wave plate, which is also called a retarder, is an optical device that alters the polarization state of a light wave passing through it. Specifically, a wave plate is an optically transparent material that resolves a beam of polarized light into two orthogonal components (i.e., at a right angle to each other); retards the phase of one component relative to the other; and recombines the components into a single beam with altered polarization characteristics. For example, a HWP shifts the polarization direction of linearly polarized light and a QWP may convert linearly polarized light to circularly polarized light and vice versa, or may convert linearly polarized light to elliptically polarized light and vice versa. Wave plates are constructed of birefringent material, for instance quartz or mica, which causes the index of refraction to differ based on the different orientations of light passing through it. The optical behavior of a wave plate varies depending on many factors, such as the thickness of the wave plate, the wavelength of incident light and the variation of the index of refraction. For instance, depending on the thickness of the wave plate, light with polarization components along both axes will be transmitted at a different polarization state. By altering these parameters, such wave plates can alter the polarization of incident light by introducing a controlled phase shift between the two polarization components of the light wave.

As shown in FIG. 3, wave plate 345 is positioned at 45 degrees, meaning that the transmission axis of the polarization component 310 (e.g., vertical polarizer) is chosen such that the linearly polarized light incident upon the wave plate is halfway (at a 45 degree angle) between the fast and slow axes of the wave plate 345. Thus, wave plate 345 converts the linear vertically polarized light to circularly polarized light. Similarly, when the reference beam is transmitted along the reference arm 370, it passes through polarization system 350 before reaching reference reflector 320. In FIG. 3, polarization system 350 is a QWP positioned at 22.5 degrees, meaning that the transmission axis of the linearly polarized light is positioned such that the fast and slow axes of the wave plate will differ, causing the light transmitted by polarization system 350 to be elliptical. Polarization system 350 may alternatively be a Faraday rotator, which is a polarization rotator based on the Faraday effect. Generally, a Faraday rotator is able to rotate the polarization state of incident light because one polarization of the incident light is in ferromagnetic resonance with the Faraday rotator material, which causes its phase velocity to be higher than the other.

When each reflected component beam is combined at the beam splitter, they form a reflected PS-OCT beam, and an interference pattern is generated. The reflected PS-OCT beam is directed to the PBS 325. As shown, PBS 325 splits the reflected PS-OCT beam into a component that is vertically polarized and directed to a vertical polarization sensitive detector 330, and a component that is horizontally polarized and directed to a horizontal polarization sensitive detector 335. In other examples, PS-OCT device 15 may implement a single-detector that can detect both reflected PS-OCT light that is vertically polarized and horizontally polarized. A single-detector system may include a combined horizontal polarization sensitive and vertical polarization sensitive detector. Single and dual-detector systems generate relatively similar reflectivity (OCT) and birefringence (PS-OCT) images, respectively.

As shown in FIG. 3, each detector may detect and generate data relating to the interference pattern, and accordingly, the reflected PS-OCT beam. This data may be transmitted to the computer 30, for example, of FIG. 2, and may be processed to generate information about the sample, including the fiber orientation and density of the tissue. Because the fiber orientation and density of the tissue affects the polarization of incident light (here, the sample beam), at least via birefringence, the phase retardation of the reflected sample beam may be determined as a function of tissue depth. This data relating to the phase retardation of the reflected sample beam, in relation to the phase retardation of the reflected reference beam, provides information directly related to the fiber orientation and density of the tissue that the sample beam passed through. Generally, tissue birefringence increases as fiber alignment, orientation and density increase, and as a result, the phase retardation of the reflected sample beam will differ as compared to the reflected reference beam.

The data generated at each of detectors 330 and 335 of PS-OCT device 15 may be transmitted to the computer 30 of FIG. 2 and used, for example, to generate a pictorial representation of the corneal tissue at the portion of the sample 322 that reflected the sample beam. Pictorial representations may be presented on a display, which may be a display screen or a heads-up display, for example. Such pictorial representations may indicate the relative fiber orientation and fiber density of the corneal tissue, based on birefringent properties of the corneal tissue at the portion of the sample reflecting the sample beam.

In free-space PS-OCT systems and PM-fiber based PS-OCT systems, the polarization state in the interferometer is maintained. Thus, wave plates are often implemented because the polarization transformations that will happen in the wave plates are known. In contrast, for single-mode fiber systems, there are unknown polarization transformations in the system itself, and therefore wave plates are typically not effective. In such single-mode fiber systems, an alternative approach is to modulate the polarization states of the light and interrogate the tissue at the same place multiple times with different polarizations and reconstruct the data generated in a way that differentiates between the tissue birefringence and the system birefringence.

Though not shown in FIG. 3, additional components may be implemented with PS-OCT device 15. For example, various circular polarizers may be used to create circularly polarized light or alternatively to selectively absorb or transmit clockwise or counter-clockwise circularly polarized light. In another example, additional wave plates, filters, polarization components, polarization systems, mirrors, or lenses may be implemented, depending on the chosen PS-OCT configuration. Also, in single-mode fiber systems, polarization modulators may often be implemented to differentiate between the polarization changes in the tissue and the system.

It should be appreciated that the PS-OCT device 15 shown in FIG. 3 is merely an illustrative configuration of a PS-OCT system. Another example PS-OCT configuration may include a PS-OCT light source that generates a PS-OCT source beam that is propagated through a polarization modulator to an interferometer, where it is split into a reference beam directed to a reference and a sample beam directed to the sample, and when the reflected sample and reflected reference beams are combined, they are directed to a detector. Another example PS-OCT configuration may include a PS-OCT light source that generates a PS-OCT source beam that is propagated to an interferometer, where it is split into a reference beam directed to a reference and a sample beam directed to the sample. The reference beam is reflected from the reference and passes through a polarization component that converts the polarization state of the returning reference beam into a polarization state with two equal orthogonal polarization components (i.e., equal vertical and horizontal components). The sample beam passes through a polarization modulator before contacting and being reflected by the sample. Both the reflected sample and reflected reference beams are combined, they are directed to a detector.

FIG. 4 illustrates an example of a process 400 for operating an ophthalmic diagnostics system in response to a stimulus. In certain embodiments, the process 400 can be implemented by any system that can process PS-OCT data. Although any number of systems, in whole or in part, can implement the process 400, to simplify discussion, the process 400 will be described in relation to example components of the ophthalmic diagnostics system 10 and the PS-OCT device 15 of FIGS. 1A-B, 2, and 3.

At block 402, the computer 30 monitors for an indication that a stimulus has been applied to a patient's eye (e.g., to the cornea or other tissue). The indication can be, for example, an automatic detection that a stimulus has been applied, a user-supplied input that a stimulus has been applied, a synchronization signal generated by the computer, an expiration of a synchronization timer for applying a stimulus, combinations of the foregoing, and/or the like. In various embodiments, indications of a stimulus may include, or be accompanied by, an indication of a location of the stimulus. The stimulus can be applied, for example, via the stimulation device 252 of FIG. 2.

At decision block 404, the computer 30 determines whether an indication of a stimulus applied to the patient's eye has been received. If no indication of a stimulus has been received, the process 400 returns to the block 402 and continues as described previously. Otherwise, if it is determined at the decision block 404 that an indication of a stimulus has been received, the process 400 proceeds to block 406.

At block 406, the computer 30 instructs the PS-OCT device 15 to emit polarized light to a desired measurement location on the patient's eye. The instruction to the PS-OCT device 15 can cause the PS-OCT device 15 to generate and emit polarized light in any of the ways described above relative to FIG. 3, and to provide PS-OCT data based thereon. In certain embodiments, as described relative to FIGS. 2 and 3, the PS-OCT data can be multichannel in nature, for example, by including data generated at each of the vertical polarization sensitive detector 330 and the horizontal polarization sensitive detector 335.

At block 408, the computer 30 receives, from the PS-OCT device 15, the PS-OCT data generated by the PS-OCT device 15 for the measurement location. The PS-OCT data can include, for example, two complex-valued OCT reflectance signals for two polarization states, e.g., one signal for each of the vertical polarization sensitive detector 330 and the horizontal polarization sensitive detector 335. Equation 2 and Equation 1 are example representations of a signal S_{V} from the vertical polarization sensitive detector 330 and a signal S_{H} from the horizontal polarization sensitive detector 335, respectively. In Equation 2, A_{V} and Ø_{V} are the amplitude and phase, respectively, of a signal captured, for example, by the vertical polarization sensitive detector 330. In Equation 1, A_{H} and Ø_{H} are the amplitude and phase, respectively, of a signal captured, for example, by the horizontal polarization sensitive detector 335.

$${S}_{V} = {A}_{V} exp ( i {∅}_{V} ) \qquad Equation 1$$

$${S}_{H} = {A}_{H} exp ( i {∅}_{H} ) \qquad Equation 2$$

At block 410, the computer 30 determines birefringent information for the patient's eye based on the PS-OCT data. The birefringent information determined at the block 410 can include various birefringent properties such as, for example, phase retardance, degree of polarization uniformity, birefringent axial orientation, combinations of the foregoing and/or the like. For example, a phase retardance R can be determined as shown in Equation 3. In other example, an axis orientation O can be determined as shown in Equation 4 below. In still another example, a degree of polarization uniformity (DOPU) can be determined according to a set of equations shown collectively as Equation 5 below.

$$R = arctan ( {A}_{H} / {A}_{V} ) \qquad Equation 3$$

$$O = ( π - {∅}_{H} - {∅}_{V} ) / 2. \qquad Equation 4$$

$${S}_{0} = {A}_{H}^{2} + {A}_{V}^{2} {S}_{1} = {A}_{H}^{2} - {A}_{V}^{2} {S}_{2} = 2 {A}_{H} {A}_{V} ( {∅}_{H} - {∅}_{V} ) {S}_{3} = 2 {A}_{H} {A}_{V} sin ( {∅}_{H} - {∅}_{V} ) OPU = \sqrt{{S}_{1}^{2} + {S}_{2}^{2} + {S}_{3}^{2}} / {S}_{0} \qquad Equation 5$$

At block 412, the computer 30 determines biomechanics information for the patient's eye based on the PS-OCT data. The biomechanics information determined at the block 412 can include various biomechanical properties such as, for example, a vibration amplitude, a vibration frequency, a quantification of tissue stiffness (e.g., Young's modulus), combinations of the foregoing and/or the like. In certain embodiments in which the PS-OCT data is multichannel in nature, for example, by including data generated at each of the vertical polarization sensitive detector 330 and the horizontal polarization sensitive detector 335 as described previously, the block 412 can include the computer 30 separately processing each channel of the PS-OCT data and determining separate corneal biomechanics for each channel (e.g., a separate vibration amplitude, a separate vibration frequency, a separate Young's modulus, etc.).

Still with reference to the block 412, in the example of FIG. 4, the computer 30 can determine the biomechanics information based on the stimulus applied to the patient's eye (e.g., to the patient's cornea). The cornea, for example, may vibrate and generate a shear wave in response to the stimulus. According to this example, the block 412 can include, for example, the computer 30 measuring vibration properties of the cornea, such as vibration frequency and vibration amplitude, which properties can be used to quantify tissue biomechanics. A vibration frequency and a vibration amplitude can be measured, for example, for each of the signal S_{V} from the vertical polarization sensitive detector 330 and the signal S_{H} from the horizontal polarization sensitive detector 335, by monitoring such signals. Temporal changes of the S_{V} and S_{H} signals at the measurement location (e.g., an A-scan location) may be recorded. These temporal changes in the signals can be analyzed by the computer 30 to extract, for each of the S_{V} and S_{H} signals, the vibration frequency and the vibration amplitude. Each vibration amplitude, for example, can be obtained for a depth location of a top surface of the cornea, or phase information from that location. Each vibration frequency, for example, can be extracted by analyzing temporal signals in the Fourier domain.

In certain embodiments, the block 412 can include the computer 30 quantifying tissue stiffness (e.g., Young's modulus) based on a speed of the shear wave mentioned above. The speed can be measured in any suitable fashion, such as with dedicated OCT scanning protocols. In various embodiments, a three-dimensional mapping of the speed of the shear wave can thus be obtained via OCT. For example, if viscosity can be neglected, Young's modulus (E) can be related to the speed of the shear wave, or shear wave speed (S_{c}), based on Equation 6 below, where ρ is the material density and my is Poisson's ratio. In general, tissue stiffness is proportional to Young's modulus and a higher Young's modulus corresponds to greater (lengthwise) stiffness.

$$E = 2 * ρ * ( 1 + m v ) * {S}_{c} \qquad Equation 6$$

At block 414, the computer 30 correlates the biomechanics information determined at the block 412 with the birefringent information determined at the block 410. For example, the computer 30 can calculate a correlation between a specific biomechanical property (or combination of properties) and a specific birefringent property (or combination of properties). The correlations can be used to evaluate, for example, the biomechanics information from the birefringent information. In this way, the computer 30 can evaluate the biomechanics information (e.g., tissue biomechanics) and offer new insights into the origins of biomechanical changes (e.g., tissue biomechanical changes).

For example, as part of the block 414, the computer 30 can compare a three-dimensional mapping of Young's modulus with three-dimensional birefringent properties such as phase retardance, axis orientation, and/or DOPU. The three-dimensional mapping can be calculated, for example, from the shear eave speed. In various embodiments, such a comparison enables the computer 30 to correlate the Young's modulus with tissue birefringence.

By way of further example, as described previously, the presence of two complex-valued OCT reflectance signals, such as the signal S_{V} from the vertical polarization sensitive detector 330 and the signal S_{H} from the horizontal polarization sensitive detector 335, respectively, can enable two vibration amplitudes and two vibration frequencies to be obtained for two different incident polarization states. In certain embodiments, the block 414 can include the computer 30 analyzing a difference between the two vibration amplitudes and, based thereon, determining if there is a relationship or correlation between the vibration amplitudes and the incident polarization states. In similar fashion, in certain embodiments, the block 414 can include, for example, the computer 30 analyzing a difference between the two vibration frequencies and, based thereon, determining if there is a relationship or correlation between the vibration frequencies and the incident polarization states.

In addition, or alternatively, the block 414 can include the computer 30 calculating a temporal sequence of tissue birefringent properties such as phase retardance, axis orientation and DOPU, for example, from each of the signals S_{V} and S_{H}. In certain embodiments, by comparing these temporal sequences, correlations between and among vibration amplitude, vibration frequency, and/or the birefringent information (e.g., birefringent properties such as, for example, phase retardance, axis orientation, and DOPU) can be identified and analyzed by the computer 30.

At block 416, the computer 30 stores and/or displays resultant data from the blocks 406-414, such as the PS-OCT data, the biomechanics information, the birefringent information, the calculated correlations, and/or data related thereto. In various embodiments, the PS-OCT data, the biomechanics information, the birefringent information, the calculated correlations, and/or data related thereto can be stored in relation to the patient in the memory 32 or other storage. In addition, or alternatively, any of the foregoing information can be displayed to a user or operator of the ophthalmic diagnostics system 10.

At decision block 418, the computer 30 determines whether to collect additional data responsive to further stimulation of the patient's eye. If it is determined at the decision block 418 to collect additional data, the process 400 returns to the block 402 and executes as described previously. Otherwise, the process 400 ends.

FIG. 5 illustrates an example of a process 500 for operating an ophthalmic diagnostics system without an external stimulus from a stimulation device. In certain embodiments, the process 500 can be implemented by any system that can process PS-OCT data. Although any number of systems, in whole or in part, can implement the process 400, to simplify discussion, the process 500 will be described in relation to example components of the ophthalmic diagnostics system 10 and the PS-OCT device 15 of FIGS. 1A-B, 2, and 3.

At block 502, the computer 30 instructs the PS-OCT device 15 to emit polarized light to a desired measurement location on the patient's eye. The instruction to the PS-OCT device 15 can cause the PS-OCT device 15 to generate and emit polarized light in any of the ways described above relative to FIG. 3, and to provide PS-OCT data based thereon. In certain embodiments, as described relative to FIGS. 2 and 3, the PS-OCT data can be multichannel in nature, for example, by including data generated at each of the vertical polarization sensitive detector 330 and the horizontal polarization sensitive detector 335.

At block 504, the computer 30 receives, from the PS-OCT device 15, the PS-OCT data generated by the PS-OCT device 15 for the measurement location. At block 506, the computer 30 determines birefringent information for the patient's eye based on the PS-OCT data. The birefringent information can be determined in any of the ways described relative to the block 410 of FIG. 4. For example, the birefringent information can include various birefringent properties such as, for example, phase retardance, degree of polarization uniformity, birefringent axial orientation, combinations of the foregoing and/or the like.

At block 508, the computer 30 determines biomechanics information for the patient's eye based on the PS-OCT data. More particularly, in certain embodiments, the biomechanics information can be determined based on the birefringent information determined at the block 406. For example, the computer 30 can use numerical algorithms such as phase decorrelation and/or intensity variance to determine, or monitor, how reflectance properties change with time. In various embodiments, the changes to reflectance properties can be used to quantify biomechanics. Advantageously, in certain embodiments, utilization of numerical algorithms in combination with the birefringent information eliminates a need for additional hardware components.

Still with reference to the block 508, in the example of FIG. 5, the computer 30 can determine the biomechanics information without an external stimulus applied to the patient's eye (e.g., to the patient's cornea). In various embodiments, the intrinsic Brownian motion, intraocular pressure and/or heart beats, for example, may introduce micro-movement in the cornea. This micro-movement may cause backscattering OCT signal change with time. In various embodiments, the biomechanics information can be obtained, at least in part, by monitoring these temporal signal changes. For PS-OCT, for example, two signal sequences can be obtained from the same measurement location (e.g., A-scan location). With these two signal sequences, a phase retardance sequence R(t), an axis orientation sequence O(t) and a DOPU sequence DOPU(t) can be calculated. In addition, or alternatively, a signal change rate for S_{H}(t) and Sᵥ(t), for example, can be used to determine, or quantify, at least a portion of the biomechanics information. At block 510, the computer 30 correlates the biomechanics information determined at the block 508 with the birefringent information determined at the block 506. For example, the computer 30 can calculate a correlation between a specific biomechanical property (or combination of properties) and a specific birefringent property (or combination of properties). The correlations can be used to evaluate, for example, the biomechanics information from the birefringent information. In this way, the computer 30 can evaluate the biomechanics information (e.g., tissue biomechanics) and offer new insights into the origins of biomechanical changes (e.g., tissue biomechanical changes).

For example, as mentioned relative to the block 508, the signal change rate for S_{H}(t) and Sᵥ(t), for example, can be used to determine, or quantify, at least a portion of the biomechanics information. Continuing this example, since two rates corresponding to two different incident polarization states are obtained, a polarization dependence of the biomechanics information can be evaluated. According to this example, because the birefringent properties and the biomechanics properties are all calculated from the S_{H}(t) and Sᵥ(t), these properties can be analyzed by the computer 30 to identify correlations. At block 512, the computer 30 stores and/or displays resultant data from the blocks 502-510, such as the PS-OCT data, the biomechanics information, the birefringent information, the calculated correlations, and/or data related thereto. In various embodiments, the PS-OCT data, the biomechanics information, the birefringent information, the calculated correlations, and/or data related thereto can be stored in relation to the patient in the memory 32 or other storage. In addition, or alternatively, any of the foregoing information can be displayed to a user or operator of the ophthalmic diagnostics system 10. After block 512, the process 500 ends.

In various embodiments, diagnostic systems such as the example diagnostic systems described herein can have various advantages. For example, such a diagnostics system that measures birefringent and biomechanical properties of diseased or healthy corneas allows for new metrics to be included in treatment planning algorithms to increase predictability and surgeon confidence. For example, corneal biomechanics such as those described herein can help evaluate therapeutic interventions in comparison with cross-linking, as well as help evaluate collagen degradation. Further, in some embodiments, corneal biomechanics is highly correlated with myopia, thereby affecting orthokeratology (Ortho-K) success (e.g., myopia reduction). High myopia may increase the risk of glaucoma. Thus, in certain embodiments, corneal biomechanics such as those described herein can help predict or identify risk of myopia and/or glaucoma.

Additionally, or alternatively, in various embodiments, diagnostic systems such as the example diagnostic systems described herein can help identify patients at risk of post-LASIK complications. Typically, refractive surgery planning uses a population-based average of corneal biomechanics. Statistically, approximately one percent of LASIK patients experience ectasia. In various embodiments, individualized corneal biomechanics as described herein may improve the ability to predict the risks of surgical intervention, such as post-LASIK ectasia.

Additionally, or alternatively, the ability to produce measurements of corneal biomechanics and apply them to treatment algorithms can support more accurate estimation, prediction, and/or establishment of cataract outcomes from patient-specific surgically induced astigmatism (SIA), Limbal Relaxing Incision (LRI) outcomes from patient-specific calculations, treatment decisions for corneal refractive power, Ortho-K outcomes, treatment and/or diagnosis of dry eye, and/or the like. For example, SIA can range from 0 to 1.5 D, which can be a meaningful source of refractive surprises. Corneal biomechanics such as those described herein can enable better prediction of a patient-specific SIA for cataract surgery and increase the accuracy of LRIs.

In certain embodiments, one general aspect includes a method of operating an ophthalmic diagnostics system. The method includes instructing a polarization sensitive optical coherence tomography device (PS-OCT) device to emit polarized light to a measurement location on eye tissue of a patient. The method also includes receiving, from the PS-OCT device, PS-OCT data for the measurement location. The method also includes determining birefringent information for the measurement location based on the PS-OCT data. The method also includes determining biomechanics information for the measurement location based on the PS-OCT data. The method also includes determining a correlation between at least a portion of the biomechanics information and at least a portion of the birefringent information.

In an example embodiment, an ophthalmic diagnostics system includes a PS-OCT device. The system also includes a computer communicably coupled to the PS-OCT device. The computer is operable to instruct the PS-OCT device to emit polarized light to a measurement location on eye tissue of a patient. The computer is also operable to receive, from the PS-OCT device, PS-OCT data for the measurement location. The computer is also operable to determine birefringent information for the measurement location based on the PS-OCT data. The computer is also operable to determine biomechanics information for the measurement location based on the PS-OCT data. The computer is also operable to determine a correlation between at least a portion of the biomechanics information and at least a portion of the birefringent information.

In an example embodiment, a method of operating an ophthalmic diagnostics system includes instructing a PS-OCT device to emit polarized light to a measurement location on eye tissue of a patient. The method also includes receiving, from the PS-OCT device, PS-OCT data for the measurement location. The method also includes determining birefringent information for the measurement location based on the PS-OCT data. The method also includes determining biomechanics information for the measurement location based on the PS-OCT data. The method also includes determining a correlation between at least a portion of the biomechanics information and at least a portion of the birefringent information.

The above-disclosed subject matter is to be considered illustrative, and not restrictive, and the appended claims are intended to cover all such modifications, enhancements, and other embodiments which fall within the true spirit and scope of the present disclosure. Thus, to the maximum extent allowed by law, the scope of the present disclosure is to be determined by the broadest permissible interpretation of the following claims and their equivalents, and shall not be restricted or limited by the foregoing detailed description.

What is claimed is:

1. An ophthalmic diagnostics system, comprising:
a polarization sensitive optical coherence tomography (PS-OCT) device;
a memory comprising executable instructions;
a processor in communication with the memory and configured to execute the instructions to:
cause the PS-OCT device to:
emit polarized light to a measurement location on eye tissue of a patient; and
generate PS-OCT data based on the polarized light,
determine birefringent information for the measurement location based on the PS-OCT data;
determine biomechanics information for the measurement location based on the PS-OCT data; and
determine a correlation between at least a portion of the biomechanics information and at least a portion of the birefringent information.

2. The ophthalmic diagnostics system of claim 1, wherein the processor is further configured to execute the instructions to receive an indication of a stimulus applied to the eye tissue, wherein the emission of polarized light to the PS-OCT device is in response to the received indication.

3. The ophthalmic diagnostics system of claim 2, wherein:
the PS-OCT device comprises a vertical polarization sensitive detector and a horizontal polarization sensitive detector;
the PS-OCT data comprises first PS-OCT data from the vertical polarization sensitive detector and second PS-OCT data from the horizontal polarization sensitive detector; and
the biomechanics information comprises first biomechanics information determined based on the first PS-OCT data and second biomechanics information determined based on the second PS-OCT data.

4. The ophthalmic diagnostics system of claim 3, wherein the first biomechanics information comprises a first vibration amplitude and the second biomechanics information comprises a second vibration amplitude.

5. The ophthalmic diagnostics system of claim 3, wherein the first biomechanics information comprises a first quantification of tissue stiffness and the second biomechanics information comprises a second quantification of tissue stiffness.

6. The ophthalmic diagnostics system of claim 1, wherein the birefringent information comprises at least one of phase retardance, degree of polarization uniformity, or birefringent axial orientation.

7. The ophthalmic diagnostics system of claim 1, wherein at least a portion of the biomechanics information is determined based on at least a portion of the birefringent information.

8. The ophthalmic diagnostics system of claim 7, wherein the determination of the biomechanics information comprises a determination of change in a reflectance property with time via at least one of phase decorrelation or intensity variance.

9. The ophthalmic diagnostics system of claim 1, wherein the processor is further configured to execute the instructions to at least one of record or display data related to the correlation.

10. The ophthalmic diagnostics system of claim 1, wherein the eye tissue comprises a cornea of the patient.

11. A method of operating an ophthalmic diagnostics system comprising a polarization sensitive optical coherence tomography device (PS-OCT) device, the method comprising:
emitting, by the ophthalmic diagnostics system, polarized light to a measurement location on eye tissue of a patient;
generating, by the ophthalmic diagnostics system, PS-OCT data for the measurement location based on the polarized light;
determining, by the ophthalmic diagnostics system, birefringent information for the measurement location based on the PS-OCT data;
determining, by the ophthalmic diagnostics system, biomechanics information for the measurement location based on the PS-OCT data; and
determining, by the ophthalmic diagnostics system, a correlation between at least a portion of the biomechanics information and at least a portion of the birefringent information.

12. The method of claim 11, further comprising receiving an indication of a stimulus applied to the eye tissue, wherein the emitting is performed in response to the received indication.

13. The method of claim 12, wherein:
the PS-OCT device comprises a first polarization sensitive detector and a second polarization sensitive detector;
the generating comprises generating first PS-OCT data by the first polarization sensitive detector and second PS-OCT data by the second polarization sensitive detector; and
the determining biomechanics information comprises determining first biomechanics information based on the first PS-OCT data and second biomechanics information based on the second PS-OCT data.

14. The method of claim 13, wherein the first biomechanics information comprises a first vibration amplitude and the second biomechanics information comprises a second vibration amplitude.

15. The method of claim 13, wherein the first biomechanics information comprises a first quantification of tissue stiffness and the second biomechanics information comprises a second quantification of tissue stiffness.

16. The method of claim 11, wherein the birefringent information comprises at least one of phase retardance, degree of polarization uniformity, or birefringent axial orientation.

17. The method of claim 11, wherein at least a portion of the biomechanics information is determined based on at least a portion of the birefringent information.

18. The method of claim 17, wherein the determining biomechanics information comprises determining change in a reflectance property with time via at least one of phase decorrelation or intensity variance.

19. The method of claim 11, further comprising at least one of recording or displaying data related to the correlation.

20. The method of claim 11, wherein the eye tissue comprises a cornea of the patient.
